(19) United States
(12) Patent Application Publication
(10) Pub. No.: US 2016/0008328 A1
(43) Pub. Date: Jan. 14, 2016

(54) Stable Pharmaceutical Package Comprising Azilsartan Medoxomil

(71) Applicant: CADILA HEALTHCARE LIMITED, Ahmedabad (IN)

(72) Inventors: BRIJ KHERA, NORTH PENNINGTON, NJ (US); NILENDU SEN, THANE (IN); SACHIN GAJANAN MOGHE, THANE (IN); NEELAM SANJEEB PARHI, THANE (IN)

(73) Assignee: CADILA HEALTHCARE LIMITED, Ahmedabad (IN)

(21) Appl. No.: 14/796,117

(22) Filed: Jul. 10, 2015

(30) Foreign Application Priority Data
Jul. 11, 2014 (IN) 2270/MUM/2014

(51) Int. Cl. A61K 31/4245; A61K 31/4035; A61K 9/20

(52) U.S. Cl. CPC A61K 31/4245; A61K 9/2009; A61K 31/4035; A61K 9/2013; A61K 9/2095

(57) ABSTRACT

The present invention relates to pharmaceutical package comprising a pharmaceutical preparation comprising azilsartan medoxomil and salts thereof, and a desiccant. Also, relates to a pharmaceutical preparation comprising azilsartan medoxomil and salts thereof and a pH modifier, wherein pH modifier provides a pH range of about 5.5 to about 6.5 when dissolved or suspended in water at a concentration of 1% at 25° C. The invention also relates to processes for the preparation of such pharmaceutical preparation and use thereof for prophylaxis or treatment of circulatory diseases.

### FIELD OF THE INVENTION

The present invention relates to pharmaceutical package comprising a pharmaceutical preparation comprising azilsartan medoxomil and salts thereof, and a desiccant. The invention also relates to processes for the preparation of such pharmaceutical package and use thereof for treatment of hypertension.

### BACKGROUND OF THE INVENTION

Hypertension affects about 20% of the adult population in developed countries. In the adult population aged 60 years or older, this percentage increases to about 60% to 70% in general. Hypertension is also associated with an increased risk of other physiological complications including stroke, myocardial infarction, atrial fibrillation, heart failure, peripheral vascular disease and renal impairment. Although a number of anti-hypertensive drugs are available in various pharmacological categories, the efficacy and safety of such drugs can vary from patient to patient and in this regard new treatments are still a desired subject.

Azilsartan, which has a chemical name as 2-ethoxy-1-((2′-(5-oxo-2,5-dihydro-1,2,4-oxadiazol-3yl)-biphenyl-4-yl)methyl)-1H-benzimidazole-7-carboxylic acid is a novel angiotensin II receptor antagonist having a chemical structure as shown in the following Formula I:

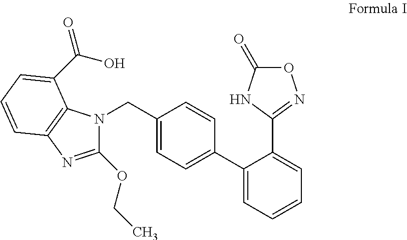 [SMILES: CCOc1nc2cccc(C(=O)O)c2n1Cc1ccc(-c2ccccc2-c2noc(=O)[nH]2)cc1]

Angiotensin II receptor antagonists are used in the management of hypertension. These antagonists may have a particular role in patients who develop cough with ACE inhibitors. Some are also used in diabetic nephropathy and in the management of heart failure. They act mainly by selective blockade of AT1 receptors, thus reducing the pressor effects of angiotension II. Known angiotension receptor II antagonists from the prior art include candesartan, eprosartan, irbesartan, losartan, olmesartan, telmisartan and valsartan.

Further, it is important that pharmaceutical products be effective and safe. A pharmaceutical product is not considered to be effective and safe if the drug is easily decomposed or denatured during storage and distribution of the pharmaceutical product, even when a pharmaceutical product was effective and safe immediately after production. Therefore, the stability of the drug is extremely important for pharmaceutical products.

Although the effectiveness and safety are most important for pharmaceutical products; however, convenience is also important from practical aspects. For example, the size, taste, smell (odor), appearance, texture and the like of the tablet, which is the most common form of a pharmaceutical product, are also important for patients taking the tablet each day.

There are many compounds which are known to produce some peculiar smell and are very unstable during storage. There is difficulty in distinguishing whether they are “compounds giving out smells themselves” or “compounds giving out smells by their decomposition”, and such distinction does not have a special meaning in the present invention (hereinafter, “compounds giving out smells themselves” and “compounds giving out smells by their decomposition” will be collectively called “compounds giving out smells”).

The compounds giving out smells are, for example, compounds having a (5-methyl-2-oxo-1,3-dioxol-4-yl)methyl group (so-called a medoxomil group) within their molecules (for example, olmesartan medoxomil and the like). The compounds having a medoxomil group within their molecules generate a low molecular weight compound, 2,3-butanedione (also called biacetyl or diacetyl), by having their medoxomil ester cleaved gradually, and 2,3-butanedione is considered to be a causative material of the peculiar smell.

As a method of decreasing an uncomfortable odor, a decomposition method, an adsorption method, a masking method and the like are known. In the decomposition method, a substance responsible for the odor is decomposed, and the method includes decomposition by ozone, decomposition by catalyst, decomposition by pharmaceutical agent and the like. In the adsorption method, a substance responsible for the odor is adsorbed, and the method includes adsorption by activated carbon, a method including adsorption to an electric field is applied with a high voltage and the like. In the masking method, aromatic and the like are used to prevent direct smell of an uncomfortable odor.

U.S. 2010/0121071 discloses a solid pharmaceutical composition comprising a compound represented by the formula (I)

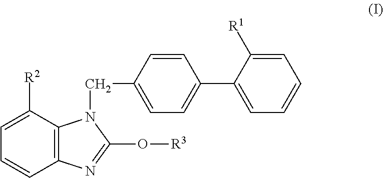

wherein R₁ is a monocyclic nitrogen-comprising heterocyclic group having a hydrogen atom that can be deprotonized, R₂ is an esterified carboxyl group, and R₃ is an optionally substituted lower alkyl, or a salt thereof, and a pH modifier.

U.S. 2012/0100093 discloses a method for reducing smells of a medicinal preparation comprising (5-methyl-2-oxo-1,3-dioxol-4-yl)methyl 2-ethoxy-1-{[2′-(5-oxo-4,5-dihydro-1,2,4-oxadiazol-3-yl)biphenyl-4-yl]methyl}-1H-benzimidazole-7-carboxylate or (5-methyl-2-oxo-1,3-dioxol-4-yl)methyl 2-cyclopropyl-1-{[2′-(5-oxo-4,5-dihydro-1,2,4-oxadiazol-3-yl)biphenyl-4-yl]methyl}-1H-benzimidazole-7-carboxylate, or a salt thereof, which comprises using calcium oxide, wherein the medicinal preparation is preserved in a medicinal package, and calcium oxide is contained in the material composing the medicinal package.

U.S. 2011/0201658 discloses a method of decreasing odor of a pharmaceutical preparation comprising 2-ethoxy-1-{[2′-(5-oxo-4,5-dihydro-1,2,4-oxadiazol-3-yl)biphenyl-4-yl]met-hyl}-1H-benzimidazole-7-carboxylic acid (5-methyl-2-oxo-1,3-dioxol-4-yl)methyl or 2-cyclopropyl-1-{[2′-(5-oxo-4,5-dihydro-1,2,4-oxadiazol-3-yl)biphenyl-4-yl]methyl}-1H-benzimidazole-7-carboxylic acid (5-methyl-2-oxo-1,3-dioxol-4-yl)methyl or a salt thereof, which comprises adding a desiccant to the pharmaceutical preparation.

U.S. 2011/0123615 discloses a solid preparation comprising a compound represented by the formula (I):

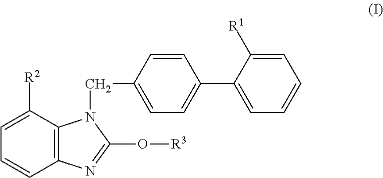

wherein R₁ is a monocyclic nitrogen-comprising heterocyclic group having a deprotonizable hydrogen atom, R₂ is an esterified carboxyl group and R₃ is an optionally substituted lower alkyl, or a salt thereof, a pH modifier and a diuretic.

A need exists in the art for methods to stabilize solid oral pharmaceutical dosage forms comprising azilsartan medoxomil and salts thereof. These stabilized pharmaceutical dosage forms would allow for longer storage periods, and would allow the amount of components to remain constant over the storage period. Also, there is a need to provide a pharmaceutical preparation comprising azilsartan medoxomil free of odor produced by hydrolysis of medoxomil group in azilsartan medoxomil.

### SUMMARY OF THE INVENTION

In one general aspect there is provided a pharmaceutical package comprising a pharmaceutical preparation comprising azilsartan medoxomil and salts thereof, and a desiccant.

In another general aspect there is provided a pharmaceutical package comprising a pharmaceutical preparation comprising azilsartan medoxomil and salts thereof, a pH modifier, and a desiccant, wherein pH modifier provides a pH range of about 5.5 to about 6.5 when dissolved or suspended in water at a concentration of 1% at 25° C.

In another general aspect there is provided a pharmaceutical package comprising a pharmaceutical preparation comprising azilsartan medoxomil and salts thereof, pH modifier and a desiccant, wherein the composition is free of odor produced by hydrolysis of (5-methyl-2-oxo-1,3-dioxol-4-yl)methyl group (i.e., a medoxomil group) in azilsartan.

In another general aspect there is provided a pharmaceutical package comprising a pharmaceutical preparation comprising azilsartan medoxomil and salts thereof, pH modifier and a desiccant, wherein the said pharmaceutical preparation retains at least 80% of the potency of azilsartan medoxomil and salts thereof in the pharmaceutical composition after storage at 40° C. and 75% relative humidity for three months.

In another general aspect there is provided a pharmaceutical package comprising a pharmaceutical preparation comprising azilsartan medoxomil and salts thereof, pH modifier and a desiccant, wherein the pharmaceutical package is a sealed package comprising a pharmaceutical preparation comprising azilsartan medoxomil and salts thereof, a pH modifier, and a desiccant.

In another general aspect there is provided a pharmaceutical package comprising a pharmaceutical preparation comprising azilsartan medoxomil and salts thereof, a diuretic, pH modifier and a desiccant.

In another general aspect there is provided a process for preparing pharmaceutical package comprising a pharmaceutical preparation comprising azilsartan medoxomil and salts thereof, pH modifier and a desiccant.

In another general aspect there is provided a method for the prophylaxis or treatment of circulatory diseases such as hypertension, cardiac failure, diabetic nephropathy, arteriosclerosis and the like, comprising administering to said subject a pharmaceutical preparation comprising azilsartan medoxomil and salts thereof, pH modifier and a desiccant.

The details of one or more embodiments of the present invention are set forth in the description below. Other features, objects and advantages of the invention will be apparent from the description.

### DETAILED DESCRIPTION OF THE INVENTION

The present inventors have achieved the stability and desired dissolution property of pharmaceutical preparation comprising azilsartan medoxomil and salts thereof after conducting extensive studies, and found that the objects can be unexpectedly accomplished by the presence of dessicant in pharmaceutical package comprising pharmaceutical preparation comprising azilsartan medoxomil and a pH modifier, wherein pH modifier provides a pH range of about 5.5 to about 6.5 when dissolved or suspended in water at a concentration of 1% at 25° C.

In addition, they have found that a pharmaceutical preparation comprising diuretic and azilsartan medoxomil and salts thereof comprising a pH modifier can be further stabilized by granulating them together with pH modifier and adjusting the pH range of a said preparation thereof to about 8 and packing said preparation along with the dessicant in the pharmaceutical package, wherein pH modifier provides a pH range of about 5.5 to about 6.5 when dissolved or suspended in water at a concentration of 1% at 25° C.

Further, it has been found by the present inventors that by changing of preservation environment during transportation or storage, increases the smells or odorous materials from the pharmaceutical preparation comprising azilsartan medoxomil and salts thereof. Further, most of smells are delivered to a nasal cavity by the moisture in headspace when the package is opened, thereby making the person feel the smells. Therefore, the present inventors have found that the odor of a preparation comprising medoxomil ester group can be decreased unexpectedly using a desiccant.

Hence, the combination of desiccant and the pharmaceutical preparation with pH modifier, wherein pH modifier provides a pH range of about 5.5 to about 6.5 when dissolved or suspended in water at a concentration of 1% at 25° C. together provides a stable pharmaceutical preparation devoid of any peculiar smell produced by the hydration of medoxomil group in azilsartan.

Moreover, such pharmaceutical preparations are also stable and may retain at least 80% of the potency of azilsartan medoxomil and salts thereof in the pharmaceutical composition after storage at 40° C. and 75% relative humidity for at least three months.

As used herein, the term azilsartan medoxomil is used in the present invention also includes a pharmacologically acceptable salt thereof. Examples of such salt include salts with inorganic bases (e.g., alkali metals such as sodium, potassium etc., alkaline earth metals such as calcium, magnesium etc., transition metals such as zinc, iron, copper etc., and the like), and organic bases (e.g., organic amines such as trimethylamine, triethylamine, pyridine, picoline, tromethamine, ethanolamine, diethanolamine, triethanolamine, dicyclohexylamine, t-butylamine, N,N′-dibenzylethylenediamine and the like, basic amino acids such as arginine, lysine, ornithine etc., and the like) and the like. The most preferable salt of azilsartan medoxomil is the potassium salt. The azilsartan medoxomil present in the pharmaceutical preparation is in an amount of about 5-30%, preferably at least 10-25% by weight.

The pH modifier to be used in the present invention is may be any as long as it simultaneously achieves stability of the preparation of azilsartan medoxomil and salts thereof and dissolution property of azilsartan medoxomil and salts thereof from the preparation and is applicable to a pharmaceutical product. In addition, plural pH modifier may be used in combination. The pH modifier to be used in the present invention has a pH about 5.5 to about 6.5. For example, an acidic substance such as tartaric acid, citric acid, lactic acid, fumaric acid, phosphoric acid, malic acid, succinic acid, ascorbic acid, acetic acid, acidic amino acid (e.g., glutamic acid, aspartic acid) and the like, an inorganic salt (e.g., alkali metal salt, alkaline earth metal salt, ammonium salt and the like) of these acidic substances, a salt of such acidic substance with an organic base (e.g., basic amino acid such as lysine, arginine and the like, meglumine and the like), a solvate (e.g., hydrate) thereof and the like are used. The pH modifier simultaneously achieves stability of a diuretic and dissolution property of the diuretic from the preparation.

The pH of the pH modifier is measured under the following conditions. To be specific, it is the pH of a solution or suspension obtained by dissolving or suspending a pH modifier in water at 25° C. at a concentration of 1 w/v %.

The pH modifier to be used in the present invention preferably affords a solution having a buffering capacity at pH above 5, such as sodium dihydrogen phosphate, monosodium citrate, a combination of citric acid and sodium ion donor and the like.

The pH modifier to be used in the present invention is preferably monosodium citrate or a combination of citric acid and sodium ion donor. In addition, citric acid and sodium hydroxide may be used in combination.

In the solid preparation of the present invention, the pH modifier present in the pharmaceutical preparation is in an amount of 0.01-20% w/w, preferably 0.05-10% w/w, more preferably 0.1-6% w/w, of the solid preparation.

Examples of the diuretic in the present invention include xanthine derivatives (e.g., theobromine sodium salicylate, theobromine calcium salicylate), thiazide preparations (e.g., ethiazide, cyclopenthiazide, trichloromethyazide, hydrochlorothiazide, hydroflumethiazide, benzylhydrochlorothiazide, penflutizide, polythiazide, methyclothiazide etc.), antialdosterone preparations (e.g., spironolactone, triamterene), carbonic anhydrase inhibitors (e.g., acetazolamide), chlorobenzenesulfonamide agents (e.g., chlorthalidone, mefruside, indapamide), azosemide, isosorbide, ethacrynic acid, piretanide, bumetanide, furosemide and the like. As the diuretic in the present invention, a chlorobenzenesulfonamide agent is preferable, and chlorthalidone and the like are more preferable. In the solid preparation of the present invention, the diuretic present in the pharmaceutical preparation is in an amount of 1-10% w/w, preferably 2-8% w/w, more preferably 3-7% w/w, of the solid preparation.

In an embodiment, the dosage form of the pharmaceutical preparation to be used in the present invention include solid dosage suitable for oral administration such as tablet, capsule, powder, granule, fine granule and the like.

In further embodiment, the pharmaceutical preparation of the present invention can be produced according to a method known per se. For example, azilsartan medoxomil and salts thereof, a pH modifier, a diuretic, additives and the like are mixed, a binder is added to the mixture to give granules, a lubricant and the like are added to the granules and the mixture is tableted into a tablet. Granules and fine granules can also be produced by a method similar to that of the tablet.

In further embodiment, the process of preparing a pharmaceutical package comprising:

a) azilsartan medoxomil and salts thereof, a pH modifier, a diuretic and additives are mixed,
b) a binder is added to the mixture of step a) to give granules,
c) a lubricant is added to the granules of step b),
d) the lubricated blend of step c) is compressed into tablets using suitable size punches,
e) the tablets of step d) is suitably packed with a dessicant.

In further embodiment, the pharmaceutical preparation of the present invention may contain pharmaceutically acceptable additives. Examples of the additive include diluent, disintegrant, binder, lubricant, surfactant, stabilizer, glidant and the like. These additives are used in an amount conventionally employed in the pharmaceutical field.

Examples of the diluent include starches such as corn starch, potato starch, wheat starch, rice starch, partly pregelatinized starch, pregelatinized starch, porous starch and the like; sugar and sugar alcohols such as lactose, fructose, glucose, mannitol (e.g., D-mannitol), sorbitol (e.g., D-sorbitol), erythritol (e.g., D-erythritol), sucrose and the like; anhydrous calcium phosphate, crystalline cellulose, microcrystalline cellulose, glycyrrhiza uralensis, sodium hydrogen carbonate, calcium phosphate, calcium sulfate, calcium carbonate, precipitated calcium carbonate, calcium silicate and the like. In the solid preparation of the present invention, the diluent present in the pharmaceutical preparation is in an amount of 10-90% w/w, preferably 25-85% w/w, more preferably 30-75% w/w, of the solid preparation.

Examples of the disintegrant include starch, cornstarch, carboxymethyl cellulose, calcium carboxymethyl cellulose, sodium carboxymethyl starch, carmellose sodium, carmellose calcium, croscarmellose sodium, crospovidone, low-substituted hydroxypropyl cellulose, hydroxypropyl starch, sodium carboxymethyl starch and the like. In the solid preparation of the present invention, the disintegrant present in the pharmaceutical preparation is in an amount of 5-35% w/w, preferably 5-30% w/w, more preferably 5-20% w/w of the solid preparation.

Examples of the binder include methyl cellulose, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums, synthetic resins and the like. In the solid preparation of the present invention, the binder present in the pharmaceutical preparation is in an amount of 1-30% w/w, preferably 1-20% w/w, more preferably 1-10% w/w of the solid preparation.

Preferable examples of the lubricant include magnesium stearate, stearic acid, calcium stearate, talc (purified talc), stearyl fumarate monosodium salt and the like. In the solid preparation of the present invention, the lubricant present in the pharmaceutical preparation is in an amount of 0.25-5% w/w, preferably 1-5% w/w of the solid preparation.

Examples of the surfactant include mono fatty acid esters of polyoxyethylene sorbitan such as those sold under the brand name Tween®; sodium lauryl sulfate, polyoxyethylene castor oil derivatives such as those sold under the brand name Cremophor®, polyethoxylated fatty acids and their derivatives, propylene glycol fatty acid esters, sterol and sterol derivatives; sorbitan fatty acid esters and their derivatives, sugar esters, polyoxyethylene-polyoxypropylene block copolymers such as those sold under the brand name Poloxamer, soy lecithin, sodium stearyl fumarate, and the like. The amount of surfactant is preferably in the range of 0.5% to 25% by weight of the composition.

Examples of the stabilizer include tocopherol, tetrasodium edetate, nicotinic acid amide, cyclodextrins and the like.

In further embodiment, the pharmaceutical preparation of present invention may be coated with a coating agent for masking of taste, enteric or sustained-release and the like. Examples of the coating agent include hydroxypropylmethylcellulose, ethylcellulose, hydroxymethylcellulose, hydroxypropylcellulose, polyoxyethyleneglycol, Tween 80, pluronic F68, cellulose acetate phthalate, hydroxypropylmethylcellulose phthalate, hydroxymethylcellulose acetate succinate, Eudragit (methacrylic acid acrylic acid copolymer, manufactured by Rohm, West Germany) and the like, and where necessary, a light shielding agent such as titanium oxide, red iron oxide and the like can also be used.

In further embodiment, the inventor of the present invention dedicatedly researched to realize that along with stable formulation there also require an efficient and continuous reduction of smell in medicinal preparations comprising azilsartan medoxomil and salts thereof giving out unpleasant smells. As a result, the inventor found that along with pharmaceutical preparation of present invention there requires desiccant to provide stability and also effectively reduced smells in a medicinal preparation.

In further embodiments, examples of the “desiccant” to be used in the present invention include activated carbon, calcium chloride, metallic oxide, such as an alkaline earth metallic oxide (e.g. calcium oxide (CaO) etc.), an alkaline earth metallic hydroxide (e.g. calcium hydroxide etc.), sulfate of an alkaline earth metal (e. g. magnesium sulfate, calcium sulfae etc.), silicon dioxide (silica gel), a bonded product of alumina oxide and silicon dioxide (silica alumina), alumina oxide (active alumina), natural or synthetic zeolite (molecular sieves 3A, 4A, SA, 13X), allophane, clay, a mixture of clay and activated carbon, a mixture of silica gel and activated carbon, a mixture of silica gel and clay, a mixture of silica alumina and activated carbon, a mixture of synthetic zeolite and activated carbon, a mixture of allophane and activated carbon (e.g., allophane added with activated carbon, or allophane kneaded with activated carbon etc.), pulp containing silica gel (e.g., ultrafine silica gel mixed between paper fibers, silica gel packaged in paper tube etc.), pulp containing calcium chloride (e.g., paper material impregnated with liquid calcium chloride, dried and coated with film etc.), pulp containing allophane (e.g., pulp impregnated with allophane liquid, dried and film coated, allophane packaged in paper tube etc.) and the like.

Only one kind of the above-mentioned desiccant may be used or two or more kinds thereof may be used in combination. In a preferred embodiment, the desiccant used in the present invention is selected from sulfate of an alkaline earth metal (e.g. magnesium sulfate, calcium sulfae etc.), a mixture of clay and activated carbon, natural or synthetic zeolite (molecular sieves 3A, 4A, SA, 13X), activated carbon, an alkaline earth metallic hydroxide (e.g. calcium hydroxide etc.), a bonded product of alumina oxide and silicon dioxide (silica alumina) and alumina oxide (active alumina).

In another embodiment, the pharmaceutical preparation comprising azilsartan medoxomil and salts thereof and a desiccant preferably coexist independently in the pharmaceutical package of the present invention. As used herein, “coexist independently” means that a pharmaceutical preparation and a desiccant exist in the same space under a physically independent condition. As long as such conditions are satisfied, they may be in contact with each other or exist separately. In addition, as used herein, the “same space” means the inside space of a bottle or blister pack, and its size is not limited as long it can afford an odor decreasing effect.

In the pharmaceutical package of the present invention, the shape of a desiccant and the configuration of co-existence of the pharmaceutical preparation and a desiccant can be appropriately selected according to the dosage form and the configuration of packaging of the pharmaceutical preparation.

For example, when the pharmaceutical preparation is tablet, capsule and the like, the desiccant can also be directly enclosed in the form of powder, granule and the like in a package container. In addition, for packaging of tablet, capsule and the like, a blister pack wherein a pharmaceutical preparation is placed in the cavity of a pan sheet generally made of a plastic or metal (e.g., aluminum etc.), and sealed with a cover sheet generally made of plastic or metal (e.g., aluminum etc.), is frequently used, where a pan sheet having further cavities for containing a desiccant in addition to the cavities for containing a pharmaceutical preparation (both cavities are not completely compartmented but have a communicating part permitting permeation of a causative substance of odor) may be formed, and a desiccant formed into a powder, granule, fine granule and the like, pellet, plate, rod, tablet and the like may be placed in the cavities for placing a desiccant and sealed with a plastic or aluminum material.

In the present invention, the “sealed package” is not particularly limited as long as it can house the preparation to be used in the present invention and a desiccant in a closed space, and includes the aforementioned package container (e.g., glass bottle, plastic bottle (polyethylene bottle etc.), a plastic bag (including one vapor-deposited with aluminum, silicon dioxide (silica) etc.), an aluminum bag, a metal can and a composite material thereof etc.), a blister pack and the like.

The amount of the desiccant to be used in the present invention is not particularly limited as long as it is sufficient to remove an odor substance, that is, an amount sufficient to suppress or decrease an odor. In addition, the amount varies depending on the kind and form of the desiccant to be used, the distance from the pharmaceutical preparation, the amount and dosage form, the volume of the space in which the pharmaceutical preparation and the desiccant are placed, the amount of the odor substance present or to be produced, the preservation conditions of the pharmaceutical preparation and the like.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

### Two Stage Granulation—Part A Azilsartan Kamedoxomil Granulated with Citric Acid Monohydrate Along with Other Exclpients and Part B Placebo Granulated with Sodium Hydroxide

| | | |
|---|---|---|
| Sr No | Ingredients | % w/w |

PART-A Granulation
Dry mix

| | | |
|---|---|---|
| 1 | Azilsartan Kamedoxomil | 23.50 |
| 2 | Mannitol (Pearlitol 25C) | 9.50 |
| 3 | MCC (Comprecel 101) | 18.63 |
| 4 | Cross Caremellose Sodium | 1.50 |
| | Binder | |
| 5 | Hydroxy Propyl Cellulose (LF) | 1.50 |
| 6 | Citric Acid Monohydrate | 3.46 |
| 7 | Purified water | q.s. |

PART-B Granulation
Dry mix

| | | |
|---|---|---|
| 8 | Mannitol (Pearlitol 25C) | 9.20 |
| 9 | MCC (Comprecel 101) | 18.50 |
| 10 | Cross Caremellose Sodium | 1.25 |
| | Binder | |
| 11 | Hydroxy Propyl Cellulose (LF) | 1.50 |
| 12 | Sodium Hydroxide | 1.86 |
| 13 | Purified water | q.s. |
| | Extra-granular | |
| 14 | MCC (Comprecel 112D) | 3.00 |
| 15 | Cross Caremellose Sodium | 5.00 |
| 16 | D&C yellow #10 Alum lake | 0.10 |
| | Lubrication | |
| 17 | Magnesium Stearate | 1.50 |
| | Total weight | 100.00 |

### Procedure:

### Part A Granulation:

1. Azilsartan Kamedoximil, mannitol, MCC and Cross Carmellose Sodium were sifted together through suitable sieve and loaded into fluid bed equipment.
2. Citric Acid Monohydrate was dissolved in purified water and granulated the dry mix of step 1 in fluid bed equipment
3. Hydroxypropyl Cellulose was dissolved in purified water and granulated the wet mass of step 2 in fluid bed equipment followed by drying.

### Part B Granulation:

4. Mannitol, MCC and Cross Carmellose Sodium were sifted together through suitable sieve and loaded into fluid bed equipment.
5. Sodium hydroxide was dissolved in purified water and granulated the dry mix of step 4 in fluid bed equipment
6. Hydroxypropyl Cellulose was dissolved in purified water and granulated the wet mass of step 5 in fluid bed equipment followed by drying.

### Milling:

7. Dried granules of step 3 and step 6 were milled through suitable screen by Quadro Co Mill.

### Extragranular Material Addition, Blending, Lubrication & Compression:

8. Microcrystalline cellulose, Cross Carmellose Sodium and D&C yellow #10 Alum lake were sifted together through suitable sieve
9. Milled material of step 7 and sifted material of step 8 were blended together for suitable time in blender
10. The step 9 material was lubricated with sifted magnesium stearate for suitable time in blender
11. The lubricated blend was compressed into tablets using suitable size punches

### Stability Data:

| | Example # 1 |
|---|---|
| Example # | 40° C./75% RH-1 M |

| Condition | HDPE without | HDPE with |
|---|---|---|
| Pack | dessicant | dessicant |
| Related substance | | |
| Impurity-1 | 1.37 | 0.38 |
| Impurity-2 | 0.12 | 0.06 |
| Impurity-6 | 2.61 | 2.09 |
| Any unknown | 0.09 | 0.00 |
| Total impurity | 4.20 | 2.58 |

### Example 2

### Two Stage Granulation—Part A Azilsartan Kamedoxomil Granulated with Citric Acid Monohydrate and Sodium Hydroxide Along with Other Excipients. Part B Placebo Granulated with Citric Acid Monohydrate and Sodium Hydroxide

| | | |
|---|---|---|
| Sr No | Ingredients | % w/w |

PART-A Granulation
Dry mix

| | | |
|---|---|---|
| 1 | Azilsartan Kamedoxomil | 23.00 |
| 2 | Mannitol (Pearlitol 25C) | 10.00 |
| 3 | MCC (Comprecel 101) | 18.13 |
| 4 | Cross Caremellose Sodium | 2.00 |
| | Binder | |
| 5 | Hydroxy Propyl Cellulose (LF) | 1.50 |
| 6 | Citric Acid Monohydrate | 0.90 |
| 7 | Sodium Hydroxide | 0.49 |
| 8 | Purified water | q.s. |

PART-B Granulation
Dry mix

| | | |
|---|---|---|
| 9 | Mannitol (Pearlitol 25C) | 9.50 |
| 10 | MCC (Comprecel 101) | 17.50 |
| 11 | Cross Caremellose Sodium | 1.95 |
| | Binder | |
| 12 | Hydroxy Propyl Cellulose (LF) | 1.50 |
| 13 | Citric Acid Monohydrate | 2.55 |
| 14 | Sodium Hydroxide | 1.38 |
| 15 | Purified water | q.s. |
| | Extra-granular | |
| 16 | MCC (Comprecel 112D) | 3.00 |
| 17 | Cross Caremellose Sodium | 5.00 |
| 18 | D&C yellow #10 Alum lake | 0.10 |
| | Lubrication | |
| 19 | Magnesium Stearate | 1.50 |
| | Weight of Core Tablet | 100.00 |

### Procedure:

### Stage 1 Granulation:

1) Azilsartan Kamedoximil, mannitol, MCC and Cross Carmellose Sodium were sifted together through suitable sieve and loaded into fluid bed equipment.
2) Citric Acid Monohydrate and Sodium hydroxide were dissolved in purified water and granulated the dry mix of step 1 in fluid bed equipment
3) Hydroxypropyl Cellulose was dissolved in purified water and granulated the wet mass of step 2 in fluid bed equipment followed by drying.

### Stage 2 Granulation:

4) Mannitol, MCC and Cross Carmellose Sodium were sifted together through suitable sieve and loaded into fluid bed equipment.
5) Citric Acid Monohydrate and Sodium hydroxide were dissolved in purified water and granulated the dry mix of step 4 in fluid bed equipment
6) Hydroxypropyl Cellulose was dissolved in purified water and granulated the wet mass of step 5 in fluid bed equipment followed by drying.

### Milling:

7) Dried granules of step 3 and step 6 were milled through suitable screen by Quadro Co Mill.

### Extragranular Material Addition, Blending, Lubrication, Compression:

8) Microcrystalline cellulose, Cross Carmellose Sodium and D&C yellow #10 Alum lake were sifted together through suitable sieve
9) Milled material of step 7 and sifted material of step 8 were blended together for suitable time in blender
10) The step 9 material was lubricated with sifted magnesium stearate for suitable time in blender
11) The lubricated blend was compressed into tablets using suitable size punches

### Stability Data:

| | |
|---|---|
| | Example # 2 |
| Example # | 40° C./75% RH-1 M |

| Condition | HDPE without | HDPE with |
|---|---|---|
| Pack | dessicant | dessicant |
| Related substance | | |
| Impurity-1 | 1.97 | 0.40 |
| Impurity-2 | 0.24 | 0.04 |
| Impurity-6 | 0.16 | 0.15 |
| Any unknown | 0.00 | 0.00 |
| Total impurity | 2.49 | 0.63 |

### Example 3

### One Stage Granulation—Azilsartan Kamedoxomil Granulated with Citric Acid Monohydrate and Sodium Hydroxide Along with Other Excipients

| | | |
|---|---|---|
| Sr No | Ingredients | % w/w |

Dry mix

| | | |
|---|---|---|
| 1 | Azilsartan Kamedoxomil | 23.00 |
| 3 | Mannitol (Pearlitol 25C) | 17.00 |
| 4 | MCC (Comprecel 101) | 33.71 |
| 5 | Cross Caremellose Sodium | 3.00 |
| | Binder | |
| 6 | Hydroxy Propyl Cellulose (LF) | 1.50 |
| 7 | Citric Acid Monohydrate | 1.11 |
| 8 | Sodium Hydroxide | 0.60 |
| 9 | Purified water | q.s. |
| | Extra-granular | |
| 10 | MCC (Comprecel 112D) | 13.48 |
| 11 | Cross Caremellose Sodium | 5.00 |
| 12 | D&C yellow #10 Alum lake | 0.10 |
| | Lubrication | |
| 13 | Magnesium Stearate | 1.50 |
| | Weight of Core Tablet | 100.00 |

### Procedure:

### Granulation:

1) Azilsartan Kamedoximil, mannitol, MCC and Cross Carmellose Sodium were sifted together through suitable sieve and loaded into fluid bed equipment.
2) Citric Acid Monohydrate and Sodium hydroxide were dissolved in purified water and granulated the dry mix of step 1 in fluid bed equipment
3) Hydroxypropyl Cellulose was dissolved in purified water and granulated the wet mass of step 2 in fluid bed equipment followed by drying.

### Milling:

4) Dried granules of step 3 were milled through suitable screen by Quadro Co Mill.

### Extragranular Material Addition, Blending, Lubrication, Compression:

5) Microcrystalline cellulose, Cross Carmellose Sodium and D&C yellow #10 Alum lake were sifted together through suitable sieve
6) Milled material of step 4 and sifted material of step 5 were blended together for suitable time in blender
7) The step 6 material was lubricated with sifted magnesium stearate for suitable time in blender
8) The lubricated blend was compressed into tablets using suitable size punches

### Stability Data:

| | |
|---|---|
| | Example # 3 |
| Example # | 40° C./75% RH-3 M |

| Condition | HDPE without | HDPE with |
|---|---|---|
| Pack | dessicant | dessicant |
| Related substance | | |
| Impurity-1 | 1.40 | 1.19 |
| Impurity-2 | 0.16 | 0.11 |
| Impurity-6 | 0.18 | 0.21 |
| Any unknown | 0.06 | 0.08 |
| Total impurity | 1.89 | 1.64 |

### Example 4

### Two Stage Granulation—Stage 1 Both APIs Granulated with Citric Acid Monohydrate and Stage 2 Placebo Granulated with Sodium Hydroxide

| | |
|---|---|
| Ingredients | % w/w |

Dry mix

| | |
|---|---|
| Azilsartan Kamedoxomil | 12.00 |
| Chlorthalidone | 6.00 |
| Mannitol (Pearlitol 25C) | 9.00 |
| MCC (Comprecel 101) | 18.40 |
| Crospovidone XL | 4.50 |

Binder

| | |
|---|---|
| Hydroxy Propyl Cellulose (LF) | 1.50 |
| Citric Acid Monohydrate | 1.81 |
| Purified water | q.s. |

Dry mix

| | |
|---|---|
| Mannitol (Pearlitol 25C) | 11.50 |
| MCC (Comprecel 101) | 22.00 |
| Crospovidone XL | 1.32 |

Binder

| | |
|---|---|
| Hydroxy Propyl Cellulose (LF) | 1.50 |
| Sodium Hydroxide | 0.97 |
| Purified water | q.s. |

Extra-granular

| | |
|---|---|
| MCC (Comprecel 112D) | 3.00 |
| Crospovidone XL | 5.00 |

Lubrication

| | |
|---|---|
| Magnesium Stearate | 1.50 |
| Weight of Core Tablet | 100.00 |

Film Coating

| | |
|---|---|
| Opadry Green (200F510022) | 2.500 |
| Purified water | q.s. |

### Procedure:

### Stage 1 Granulation:

1) Azilsartan Kamedoximil, chlorthalidone, mannitol, MCC and Crospovidone XL were sifted together through suitable sieve and loaded into fluid bed equipment.
2) Citric Acid Monohydrate was dissolved in purified water and granulated the dry mix of step 1 in fluid bed equipment
3) Hydroxy propyl Cellulose was dissolved in purified water and granulated the wet mass of step 2 in fluid bed equipment followed by drying.

### Stage 2 Granulation:

4) Mannitol, MCC and Crospovidone XL were sifted together through suitable sieve and loaded into fluid bed equipment.
5) Sodium hydroxide was dissolved in purified water and granulated the dry mix of step 4 in fluid bed equipment
6) Hydroxypropyl Cellulose was dissolved in purified water and granulated the wet mass of step 5 in fluid bed equipment followed by drying.

### Milling:

7) Dried granules of step 3 and step 6 were milled through suitable screen by Quadro Co Mill.

### Extragranular Material Addition, Blending, Lubrication, Compression and Coating:

8) Microcrystalline cellulose, Crospovidone XL were sifted together through suitable sieve
9) Milled material of step 7 and sifted material of step 8 were blended together for suitable time in blender
10) The step 9 material was lubricated with sifted magnesium stearate for suitable time in blender
11) The lubricated blend was compressed into tablets using suitable size punches
12) Opadry was dispersed in appropriate quantity of purified water and stirred to get homogeneous suspension.
13) The tablets of step 10 were coated in Coater using step 12 dispersion till desired weight gain was obtained.

### Stability Data:

| | |
|---|---|
| Example # | Example # 4 |
| Condition | 40° C./75% RH-1 M |

| Pack | HDPE with dessicant |
|---|---|
| Related substance | |
| Impurity-1 | 0.51 |
| Impurity-2 | 0.12 |
| Impurity-6 | 1.05 |
| Any unknown | 0.03 |
| Total impurity | 1.83 |

### Example 5

### One Stage Granulation—Both APIs Granulated with Citric Acid Monohydrate and Sodium Hydroxide

| Sr No | Ingredients | % w/w |
|---|---|---|
| | Dry mix | |
| 1 | Azilsartan Kamedoxomil | 11.00 |
| 9 | Chlorthalidone | 7.00 |
| 2 | Mannitol (Pearlitol 25C) | 18.00 |
| 3 | MCC (Comprecel 101) | 32.80 |
| 4 | Crospovidone XL | 5.00 |
| | Binder | |
| 5 | HPMC 5 CPS | 1.50 |
| 6 | Citric Acid Monohydrate | 0.90 |
| 7 | Sodium Hydroxide | 0.49 |
| 8 | Purified water | q.s. |
| | Extra-granular | |
| 15 | MCC (Comprecel 112D) | 16.81 |
| 16 | Crospovidone XL | 5.00 |
| | Lubrication | |
| 17 | Magnesium Stearate | 1.50 |
| | Weight of Core Tablet | 100.00 |
| | Film Coating | |
| 18 | Opadry Green (03F510038) | 2.500 |
| 19 | Purified water | q.s. |

### Procedure:

### Granulation:

1) Azilsartan Kamedoximil, Chlorthalidone, mannitol, MCC and Crospovidone XL were sifted together through suitable sieve and loaded into fluid bed equipment.
2) Citric Acid Monohydrate and Sodium hydroxide were dissolved in purified water and granulated the dry mix of step 1 in fluid bed equipment
3) HPMC 5 CPS was dissolved in purified water and granulated the wet mass of step 2 in fluid bed equipment followed by drying.

### Milling:

4) Dried granules of step 3 were milled through suitable screen by Quadro Co Mill.

### Extragranular Material Addition, Blending, Lubrication, Compression and Coating:

5) Microcrystalline cellulose, Crospovidone XL were sifted together through suitable sieve
6) Material of step 4 was milled and sifted material of step 5 were blended together for suitable time in blender
7) The step 6 material was lubricated with sifted magnesium stearate for suitable time in blender
8) The lubricated blend was compressed into tablets using suitable size punches
9) Opadry was dispersed in appropriate quantity of purified water and stirred to get homogeneous suspension.
10) The tablets of step 8 were coated in Coater using step 9 dispersion till desired weight gain was obtained.

### Stability Data:

| | Condition | |
|---|---|---|
| | Initial | 40° C./75% RH-1 Month |
| Impurity | Pack | HDPE with Desiccant |
| Impurity-1 | 0.48 | 0.49 |
| Impurity-2 | — | 0.05 |
| Impurity-6 | 0.09 | 0.11 |
| Any Unknown | BQL | 0.07 |
| Total Impurity | 0.60 | 0.79 |

### Example 6

### Two Stage Granulation—Azilsartan Kamedoxomil Granulated with Citric Acid Monohydrate Along with Other Excipients and Placebo Granulated with Sodium Hydroxide

| Sr. No | Ingredients | % w/w |
|---|---|---|
| | Dry mix (Part A Granulation) | |
| 1 | Azilsartan Kamedoxomil | 23.71 |
| 2 | Mannitol (Pearlitol 25C) | 11.86 |
| 3 | MCC (Comprecel 101) | 17.68 |
| 4 | Cross carmellose sodium | 1.50 |
| | Binder | |
| 5 | Hydroxy Propyl Cellulose (LF) | 1.50 |
| 6 | Citric Acid Monohydrate | 1.44 |
| 7 | Purified water | q.s. |
| | Dry mix (Part B Granulation) | |
| 8 | Mannitol (Pearlitol 25C) | 10.50 |
| 9 | MCC (Comprecel 101) | 18.33 |
| 10 | Cross carmellose sodium | 1.50 |
| | Binder | |
| 11 | Hydroxy Propyl Cellulose (LF) | 1.50 |
| 12 | Sodium Hydroxide | 0.78 |
| 13 | Purified water | q.s. |
| | Extra-granular | |
| 14 | MCC (Comprecel 112D) | 3.00 |
| 15 | Cross carmellose sodium | 5.00 |
| 16 | Lake Blend LB-520004 Yellow | 0.20 |
| | Lubrication | |
| 17 | Magnesium Stearate | 1.50 |
| | Total weight | 100.00 |

### Procedure:

### Stage 1 Granulation:

1. Azilsartan Medoxomil Potassium, Mannitol, MCC and Croscarmellose sodium were sifted together through suitable sieve and loaded into fluid bed equipment.
2. HPC and citric acid monohydrate were dissolved in purified water and granulated the dry mix of step 1 in fluid bed equipment followed by drying

### Stage 2 Granulation:

3. Mannitol, MCC and Croscarmellose sodium were sifted together through suitable sieve and loaded into fluid bed equipment.
4. Sodium hydroxide was dissolved in purified water and granulated the wet mass of step 3 in fluid bed equipment.
5. HPC was dissolved in purified water and granulated the wet mass step 4 in fluid bed equipment followed by drying

### Milling:

6. Dried granules of step 2 and step 5 were milled through suitable screen by Quadro Co Mill.

### Extragranular Material Addition, Blending, Lubrication, Compression and Coating:

7. Microcrystalline cellulose, Crospovidone XL and Lake blend Yellow were sifted together through suitable sieve
8. Milled material of step 6 and sifted material of step 7 was blended together for suitable time in blender
9. The material of step 8 was lubricated with sifted magnesium stearate for suitable time in blender
10. The lubricated blend was compressed into tablets using suitable size punches

### Example 7

### Two Stage Granulation—Azilsartan Granulated with Citric Acid Monohydrate and Sodium Hydroxide and Chlorthalidone Granulated with Binder Solution

| Sr No | Ingredients | % w/w |
|---|---|---|
| | Dry mix (Part A Granulation) | |
| 1 | Chlorthalidone | 6.78 |
| 2 | Mannitol (Pearlitol 25C) | 28.32 |
| 3 | MCC (Comprecel 101) | 2.44 |
| | Binder | |
| 4 | Hydroxy Propyl Cellulose (LF) | 1.46 |
| 5 | Purified water | q.s. |
| | Dry mix (Part B Granulation) | |
| 6 | Azilsartan Kamedoxomil | 11.57 |
| 7 | Mannitol (Pearlitol 25C) | 24.91 |
| 8 | MCC (Comprecel 101) | 2.44 |
| | Binder | |
| 9 | Hydroxy Propyl Cellulose (LF) | 1.46 |
| 10 | Citric Acid Monohydrate | 0.88 |
| 11 | Sodium Hydroxide | 0.47 |
| 12 | Purified water | q.s. |
| | Extra-granular | |
| 13 | MCC (Comprecel 112D) | 9.76 |
| 14 | Crospovidone XL | 6.10 |
| | Lubrication | |
| 15 | Magnesium Stearate | 0.98 |
| | Film Coating | |
| 16 | Opadry Green (03F510038) | 2.44 |
| 17 | Purified water | q.s. |
| | Total weight | 100.000 |

### Procedure:

### Stage 1 Granulation:

1. Chlorthalidone, Mannitol and MCC were sifted together through suitable sieve and loaded into fluid bed equipment.
2. HPC was dissolved in purified water and granulated the dry mix of step 1 in fluid bed equipment followed by drying

### Stage 2 Granulation:

3. Azilsartan Medoxomil Potassium, Mannitol and MCC were sifted together through suitable sieve and loaded into fluid bed equipment.
4. Citric acid monohydrate and sodium hydroxide were dissolved in purified water and granulated the wet mass of step 3 in fluid bed equipment.
5. HPC was dissolved in purified water and granulate the wet mass step 4 in fluid bed equipment followed by drying

### Milling:

6. Dried granules of step 2 and step 5 were milled through suitable screen by Quadro Co Mill.

### Extragranular Material Addition, Blending, Lubrication, Compression and Coating:

7. Microcrystalline cellulose, Crospovidone XL were sifted together through suitable sieve
8. Milled material of step 6 and sifted material of step 7 were blended together for suitable time in blender
9. The material of step 8 was lubricated & sifted with magnesium stearate for suitable time in blender
10. The lubricated blend was compressed into tablets using suitable size punches
11. Opadry was dispersed in appropriate quantity of Purified water and stir to get homogeneous suspension.
12. The tablets were coated in Coater using step 11 dispersion till desired weight gain was obtained.

### Stability Data:

| | Condition | | |
|---|---|---|---|
| | | 50° C./80% | 50° C./80% |
| | | RH-15 days | RH-15 days |
| | Initial | HDPE with Desiccant | HDPE with Desiccant |
| Impurity | Pack | (Molecular Sieve) | (Activated Clay) |
| Impurity-1 | 0.24 | 0.18 | 0.33 |
| Impurity-2 | — | — | — |
| Impurity-6 | 0.13 | 0.14 | 0.16 |
| Any Unknown | 0.03 | 0.02 | 0.04 |
| Total Impurity | 0.46 | 0.42 | 0.60 |

We claim:

1. A pharmaceutical package comprising a pharmaceutical preparation comprising azilsartan medoxomil and salts thereof, and a desiccant.

2. The pharmaceutical package as claimed in claim 1, the pharmaceutical preparation comprising azilsartan medoxomil and salts thereof, a pH modifier, and a desiccant, wherein pH modifier provides a pH range of about 5.5 to about 6.5 when dissolved or suspended in water at a concentration of 1% at 25° C.

3. The pharmaceutical package as claimed in claim 1, the pharmaceutical preparation comprising azilsartan medoxomil and salts thereof, pH modifier and a desiccant, wherein the composition is free of odor produced by hydrolysis of (5-methyl-2-oxo-1,3-dioxol-4-yl)methyl group (i.e., a medoxomil group) in azilsartan.

4. The pharmaceutical package as claimed in claim 1, the pharmaceutical preparation comprising azilsartan medoxomil and salts thereof, pH modifier and a desiccant, wherein the said pharmaceutical preparation retains at least 80% of the potency of azilsartan medoxomil and salts thereof in the pharmaceutical composition after storage at 40° C. and 75% relative humidity for three months.

5. The pharmaceutical package as claimed in claim 1, the pharmaceutical preparation comprising azilsartan medoxomil and salts thereof, pH modifier and a desiccant, wherein the pharmaceutical package is a sealed package comprising a pharmaceutical preparation comprising azilsartan medoxomil and salts thereof, a pH modifier, and a desiccant.

6. The pharmaceutical package as claimed in claim 1, the desiccant is selected from the group comprising activated carbon, calcium chloride, metallic oxide, such as an alkaline earth metallic oxide, an alkaline earth metallic hydroxide, sulfate of an alkaline earth metal, silicon dioxide (silica gel), a bonded product of alumina oxide and silicon dioxide, alumina oxide, natural or synthetic zeolite (molecular sieves 3A, 4A, SA, 13X), allophane, clay, a mixture of clay and activated carbon, a mixture of silica gel and activated carbon, a mixture of silica gel and clay, a mixture of silica alumina and activated carbon, a mixture of synthetic zeolite and activated carbon, a mixture of allophane and activated carbon, pulp containing silica, pulp containing calcium chloride, pulp containing allophane.

7. The pharmaceutical package as claimed in claim 2, the pH modifier is selected from the group comprising an acidic substance (such as tartaric acid, citric acid, lactic acid, fumaric acid, phosphoric acid, malic acid, succinic acid, ascorbic acid, acetic acid), acidic amino acid (e.g., glutamic acid, aspartic acid), an inorganic salt (e.g., alkali metal salt, alkaline earth metal salt, ammonium salt and the like) of these acidic substances, a salt of such acidic substance with an organic base (e.g., basic amino acid such as lysine, arginine, meglumine).

8. The pharmaceutical package as claimed in claim 1, further comprises a diuretic and salts thereof.

9. The pharmaceutical package as claimed in claim 8, the diuretic is selected from a group comprising xanthine derivatives (e.g., theobromine sodium salicylate, theobromine calcium salicylate), thiazide preparations (e.g., ethiazide, cyclopenthiazide, trichloromethyazide, hydrochlorothiazide, hydroflumethiazide, benzylhydrochlorothiazide, penflutizide, polythiazide, methyclothiazide etc.), antialdosterone preparations (e.g., spironolactone, triamterene), carbonic anhydrase inhibitors (e.g., acetazolamide), chlorobenzenesulfonamide agents (e.g., chlorthalidone, mefruside, indapamide), azosemide, isosorbide, ethacrynic acid, piretanide, bumetanide, furosemide).

10. The pharmaceutical package as claimed in claim 1, further comprises additives selected from the group comprising diluent, disintegrant, binder, lubricant, surfactant, stabilizer, and glidant.

11. The process of preparing a pharmaceutical package comprising:
a) azilsartan medoxomil and salts thereof, a pH modifier, a diuretic and additives are mixed,
b) a binder is added to the mixture of step a) to give granules,
c) a lubricant is added to the granules of step b),
d) the lubricated blend of step c) is compressed into tablets using suitable size punches,
e) the tablets of step d) is suitably packed with a dessicant.
